(19) United States
(12) Patent Application Publication
(10) Pub. No.: US 2006/0121122 A1
(43) Pub. Date: Jun. 8, 2006

(54) Process for producing microcapsule

(75) Inventors: Mitsutoshi Nakajima, Tsukuba-shi (JP); Tatsuya Oda, Kashiwa-shi (JP); Shinji Sugiura, Tsukuba-shi (JP)

Correspondence Address: CARRIER BLACKMAN AND ASSOCIATES, 24101 NOVI ROAD, SUITE 100, NOVI, MI, 48375, US

(73) Assignee: Yuu Koyama, Tokyo (JP)

(21) Appl. No.: 10/525,108

(22) PCT Filed: Sep. 17, 2003
(86) PCT No.: PCT/JP03/11846

§ 371 Date: Sep. 15, 2005

(30) Foreign Application Priority Data
Sep. 18, 2002 (JP) 2002-271973

(51) Int. Cl. A61K 9/50; B01J 13/04

(52) U.S. Cl. 424/490; 264/4.1

(57) ABSTRACT

A polyelectrolyte solution as a disperse phase is fed into one of the chambers which are partitioned by a plate having a plurality of narrow holes (microchannels), a continuous phase is fed into the other chamber, and pressure is applied to the disperse phase forcing it through the holes into the continuous phase so as to prepare an emulsion. This emulsion is demulsified, and at the same time the disperse phase is brought into contact with a polyelectrolyte solution having a reverse electric charge to the disperse phase or a polyvalent ion solution, and a gel layer is formed around the spherical disperse phase by a polyelectrolyte reaction. Thereby, a double-structured capsule is obtained, in which the outside is insoluble gel and the inside is a polyelectrolyte solution to which a cell has been added.

### TECHNICAL FIELD

The present invention relates to a manufacturing method for microcapsules which are used in a DDS (drug delivery system), a food industry, or cosmetic manufacturing.

### BACKGROUND ART

As a capsule to be transplanted into a body, there has been known a microcapsule of 500-800 μm in which one or two cell(s) (islands of Langerhans) is encapsulated. (Document, “protein, nucleic acid, enzyme Vol. 45, No. 13” 2000)

In this capsule, an outside hydrogel functions as a barrier to an attack from an immune mechanism (rejection reaction), and thereby the islands of Langerhans can secrete insulin for a long period of time in the body.

The first proposal regarding such a capsule was made in U.S. Pat. No. 4,352,883 (1979). This known art material describes that a cell is fixed in calcium alginate gel.

As a technique for fixing a cell inside a shell which endures an attack from an immune mechanism and transplanting into a body, there have also been known Japanese Patent Application Publication No. 10-500889, Japanese Patent Application Publication No. 11-130698, and Japanese Patent Application Publication No. 2002-507473.

Japanese Patent Application Publication No. 10-500889 has disclosed that a rotavirus is encapsulated in a microcapsule, the outside shell of which is made by a reaction of alginic acid and spermine, and the inside of which is an aqueous core.

Japanese Patent Application Publication No. 11-130698 has disclosed that an alginic acid aqueous solution (W) is emulsified in fatty acid ester (O) so as to produce a W/O emulsion, polyvalent metal (Ca²⁺ or Ba²⁺) is added to the emulsion so as to form primary particles of alginic acid polyvalent metal salt (gel) having a diameter of 0.01-5 μm, and a poorly soluble medicine is carried by the aggregate of the primary particles.

Japanese Patent Application Publication No. 2002-507473 has disclosed that particles of an alginic acid aqueous solution are prepared by atomizing, and microcapsules of 100-400 μm are obtained by allowing the particles of an alginic acid aqueous solution to collide with a Ca²⁺ solution flowing down in a film shape.

In addition, Japanese Patent Application Publication No. 09-500132 has proposed a vaccine having a size of 15 μm or less for oral delivery in which a hydrogel is used to encapsulate.

The above-mentioned outside shell (gel) is formed by a polyelectrolyte reaction. Specifically, a poly anion solution such as an alginic acid solution is dropped onto a poly cation solution by using a nozzle as disclosed in “Biotechnology Progress 13, 562-568, 1997”.

Also, a method using a double nozzle in order to reduce the diameter of a capsule has been disclosed in “AICHE J, 40, 1026-1031, 1994”. In this method, a capsule of 2 mm-200 μm is prepared by feeding a polyelectrolyte solution from an inner nozzle and feeding air from an outer nozzle.

According to the above-mentioned conventional methods, it is possible to obtain microcapsules having a diameter in the range of from 0.01 μm to several hundreds of μm. However, in the conventional methods, the distribution of the particle diameter is wide, that is, it is difficult to obtain microcapsules having a uniform diameter.

Specifically, in Japanese Patent Application Publication No. 10-500889, and Japanese Patent Application Publication No. 2002-507473, an alginic acid solution is atomized into the air so as to make small particles, and then the particles are brought into contact with a Ca²⁺ aqueous solution. However, in such methods, capsules having a uniform diameter cannot be obtained.

In Japanese Patent Application Publication No. 11-130698, a W/O emulsion is produced by a conventional method, and this emulsion is brought into contact with a Ca²⁺ aqueous solution. In this case, however, it is difficult to control the diameter of the droplets of the disperse phase within a certain range. Accordingly, although a very fine particle can be produced, it is impossible to produce a capsule having a double structure in which an aqueous solution is encapsulated inside and the outside shell is gel.

The above-mentioned documents suggest that a microcapsule encapsulating a cell can be transplanted in a body so as to function as “a micro medicine factory”. For this purpose, the cell needs to not only secrete an effective material such as insulin or an antineoplastic agent but also be alive in the microcapsule for a long period of time.

In order to allow the cell to be alive in the microcapsule for a long period of time, the particle diameter of the microcapsule is an important factor.

Specifically, in the microcapsule for encapsulating a cell, the outside shell (gel) needs to not only endure an attack from an immune mechanism but also release a secretion from the cell, take nutrition necessary for the cell to keep alive, and excrete waste products generated in the capsule.

According to the present inventors' research, when the radius of the microcapsule is more than 150 μm (diameter: 300 μm), nutrition cannot be fed to the cell fixed in the center, and waste products cannot be excreted from the cell. Consequently, the cell dies. In contrast, if the diameter of the microcapsule is too small, it is impossible to fix a cell inside.

Therefore, most of microcapsules must have a diameter within an extremely limited range.

As for microcapsules for encapsulating a cell, the diameter distribution must be within a narrow range of 50-300 μm. Although a conventional method in which dropping is used can manufacture a microcapsule having a diameter within the above-mentioned range, it is impossible to manufacture microcapsules having a uniform diameter. Also, in a conventional method which uses an emulsion obtained by simple stirring, it is impossible to manufacture microcapsules having a uniform diameter within a certain range.

Incidentally, microcapsules having a uniform particle diameter are required in other fields such as food or cosmetic.

### DISCLOSURE OF THE INVENTION

In order to solve the above-mentioned problems, according to the present invention, there is provided a manufacturing method for microcapsules comprising the steps of preparing an emulsion which contains a polyelectrolyte solution as a disperse phase having a uniform diameter, demulsifying the emulsion, and contacting the polyelectrolyte solution as a disperse phase with a polyelectrolyte solution having a reverse electric charge to the polyelectrolyte solution as a disperse phase or a polyvalent ion solution at the same time of the demulsifying step so as to form a gel layer made of a polyelectrolyte complex around fine particles of the polyelectrolyte solution as a disperse phase by a polyelectrolyte reaction.

In the present invention, a polyelectrolyte solution is turned into an emulsion which contains a disperse phase having a uniform diameter without directly contacting the polyelectrolyte solution with another polyelectrolyte solution having a reverse electric charge thereto or a polyvalent ion solution, and thereafter the emulsion is brought into contact with a polyelectrolyte solution having a reverse electric charge or a polyvalent ion solution. As a result of this, it is possible to obtain microcapsules having substantially the same diameter as the disperse phase.

In order to obtain microcapsules having a uniform diameter, it is necessary to obtain an emulsion, the disperse phase of which has a uniform diameter. For this purpose, preferably, the disperse phase and the continuous phase are separated by a plate having penetrating holes, and the disperse phase is pushed into the continuous phase as microspheres by applying greater pressure to the disperse phase than to the continuous phase.

Also, in order to contact the disperse phase with a polyelectrolyte solution having a reverse electric charge or a polyvalent ion solution efficiently, it is necessary to demulsify the emulsion. There are two methods for demulsifying. The first one is a method in which the concentration of a surface-active agent, which is commonly added to a continuous phase to keep the emulsion state, is reduced by adding the same material as the continuous phase (such as hexane) or a soluble material to the continuous phase. The second one is a method in which a surface-active agent is originally not added at the time of preparing the emulsion. In the second method, since the emulsion is demulsified in a short period of time, the contacting step must be conducted immediately.

Examples of the disperse phase include an alginic acid, carboxymethyl cellulose, pectin, carrageenan, sulfate cellulose, and chondroitin sulfuric acid. Examples of the polyelectrolyte to be reacted with the disperse phase include a polyamino acid (such as polyhistidine, polylysine, or polyornithine), polymer containing a primary amine group, a secondary amine group, a tertiary amine group, or pyridinyl nitrogen (such as polyethylene imine, polyallyl imine, polyether amine, or polyvinyl pyridine), and aminated polysaccharide (such as chitosan). Examples of the polyvalent ion to be reacted with the disperse phase include Ca²⁺, Ba²⁺, Pb²⁺, Cu²⁺, Cd²⁺, Sr²⁺, Co²⁺, Ni²⁺, Zn²⁺ and Mn²⁺.

### BRIEF DESCRIPTION OF THE DRAWINGS

FIGS. 1(a)-(c) show an emulsion preparing step of a manufacturing method for microcapsules according to the present invention;

FIGS. 2(a) and (b) show manufacturing of microcapsules according to the present invention;

FIG. 3 is an enlarged cross-sectional view of a microcapsule obtained by the method according to the present invention;

FIG. 4 is a cross-sectional view of an apparatus for preparing an emulsion which is used in Examples 1 and 2;

FIG. 5 is a photomicrograph showing a state of preparing an emulsion in Example 1;

FIG. 6 is a photomicrograph of a microcapsule obtained in Example 1,

FIG. 7 is a photomicrograph showing a state of preparing an emulsion in Example 2; and

FIG. 8 is a photomicrograph of a microcapsule obtained in Example 2,

### BEST MODE FOR CARRYING OUT THE INVENTION

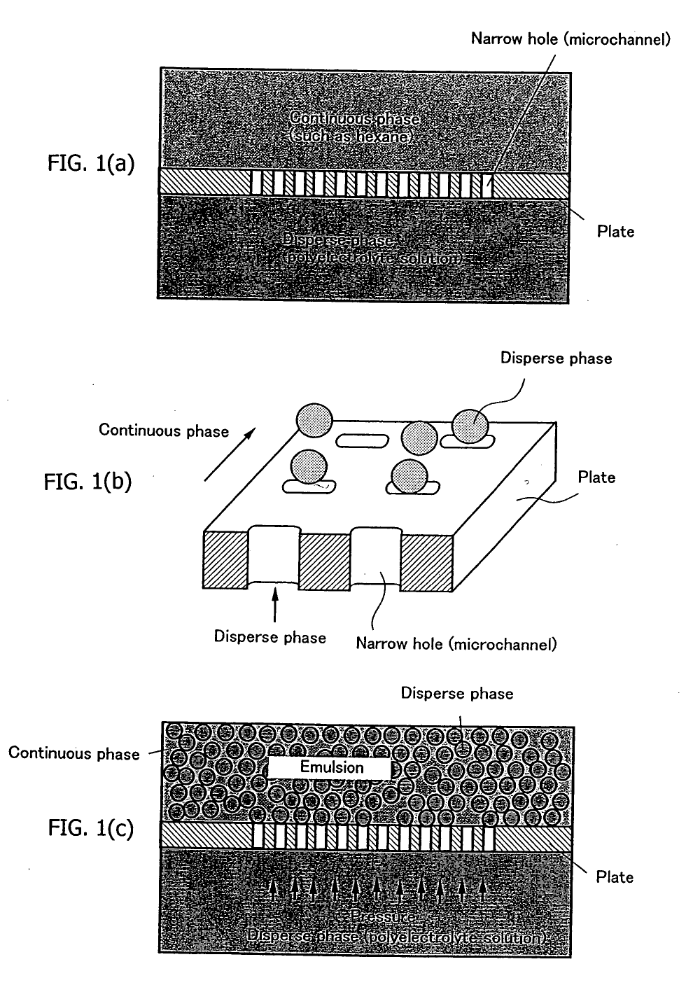

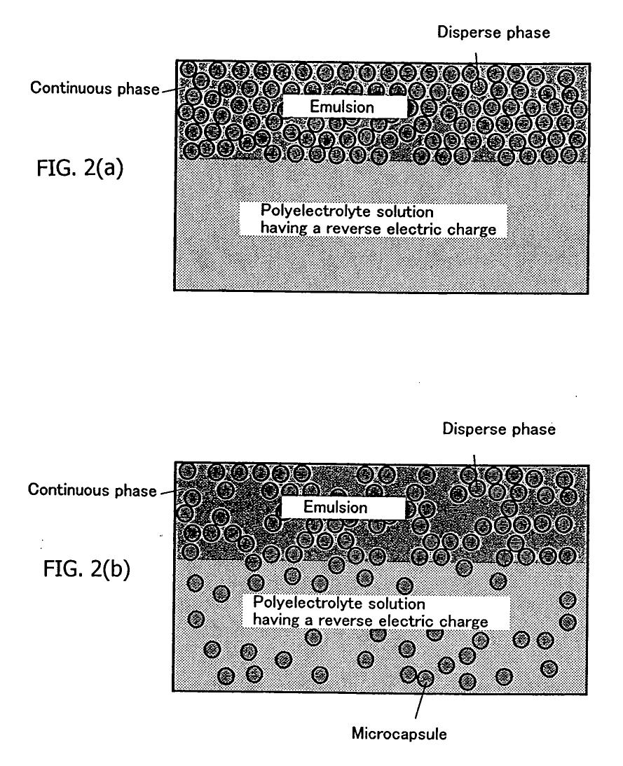

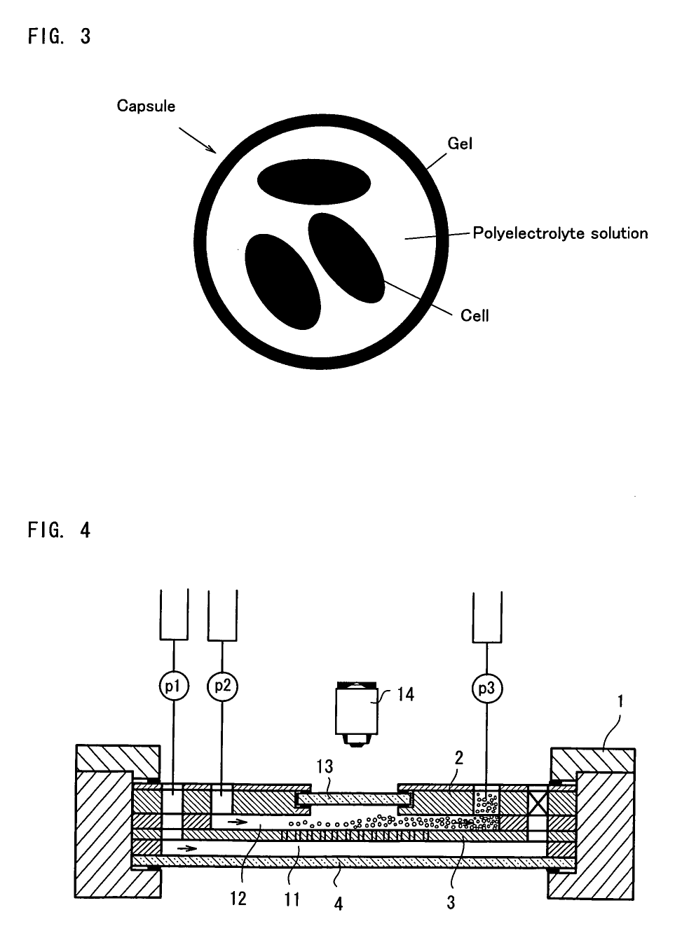

Embodiments of the present invention will now be described with reference to the attached drawings. FIGS. 1(a)-(c) show an emulsion preparing step of a manufacturing method for microcapsules according to the present invention, FIGS. 2(a) and (b) show manufacturing of microcapsules according to the present invention, and FIG. 3 is an enlarged cross-sectional view of a microcapsule obtained by the method according to the present invention.

As shown in FIG. 1(a), a polyelectrolyte solution as a disperse phase is fed into one of the chambers which are partitioned by a plate having a plurality of narrow holes, and a continuous phase (hexane) is fed into the other chamber.

Next, pressure is applied to the polyelectrolyte solution. Then, the polyelectrolyte solution enters the continuous phase while turning into a disperse phase as shown in FIG. 1(b), and an emulsion is prepared as shown in FIG. 1(c).

The shape of the disperse phase is spherical. The diameter of the spherical disperse phase depends on the size of the holes. If the size of the holes is uniform, the diameter of the obtained disperse phase becomes uniform. The holes are formed by plasma etching which is used for manufacturing an integrated circuit. In addition, a more uniform disperse phase can be obtained by making the shape of the hole non-circular.

The emulsion prepared in the above-mentioned manner is put on a polyelectrolyte solution having a reverse electric charge to the disperse phase or a polyvalent ion solution within a single vessel in a state of keeping the phase separation as shown in FIG. 2(a), and thereafter the emulsion is demulsified.

The emulsion is demulsified by adding the same material as the continuous phase (hexane) or a soluble material to the continuous phase (such as soybean oil, triolein, or octane) to the emulsion so as to reduce the concentration of the surface-active agent in the continuous phase, or by originally not adding a surface-active agent to the continuous phase.

When the emulsion has been demulsified, the disperse phase is contacted and reacted with the polyelectrolyte solution having a reverse electric charge to the disperse phase or the polyvalent ion solution, and a gel layer is formed around the spherical disperse phase. Finally, as shown in FIG. 3, a double-structured capsule is obtained, in which the outside is insoluble gel and the inside is a polyelectrolyte solution to which a cell has been added.

The microcapsule encapsulating a cell can be used for a medical treatment of a human body or a prevention against disease. In such a case, the microcapsule is injected into the parts of a human body by an injector, a catheter or an operation.

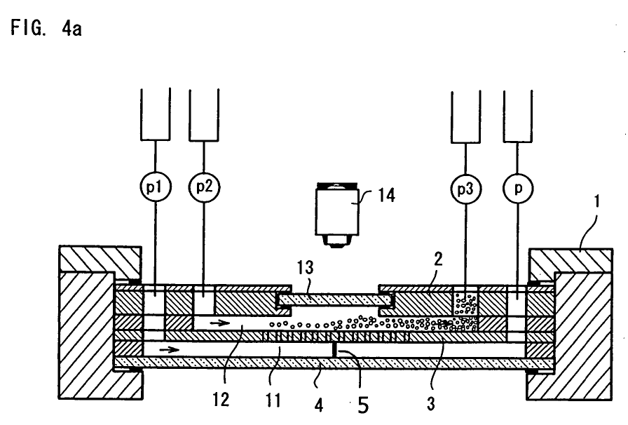

Next, embodiments of the present invention will be explained. FIG. 4 is a cross-sectional view of an apparatus for preparing an emulsion which is used in Examples 1 and 2. The apparatus for preparing an emulsion is comprised of an annular case 1, and plates 2, 3, 4 and spacers which are assembled within the case 1. The disperse phase flows through a liquid-sealed first passage 11, and the continuous phase and the emulsion flows through a liquid-sealed second passage 12. The first passage 11 and the second passage 12 are connected by narrow holes (microchannels) which are provided in the intermediate plate 3. P1 is a feeding pump for the disperse phase, P2 is a feeding pump for the continuous phase, and P3 is a withdrawing pump for the emulsion. A transparent window 13 and a CCD camera are also provided in the apparatus.

### EXAMPLE 1

Chitosan (manufactured by KIMICA Corporation) and sodium carboxymethyl cellulose (manufactured by Nippon Rika Co., Ltd.) were employed as a raw material of the capsule. Hexane was used as a continuous phase, and TGCR-310 (manufactured by Sakamoto Yakuhin Kogyo Co., Ltd.) was used as a surface-active agent.

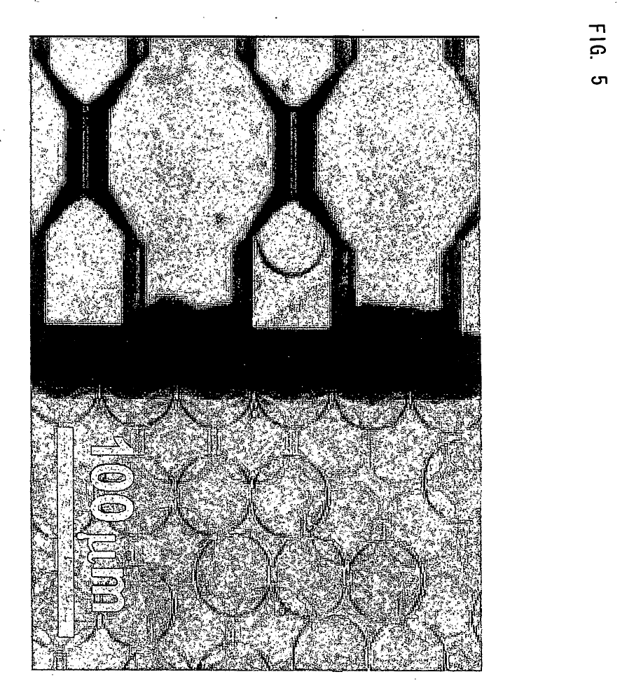

Carboxymethyl cellulose of 0.8 wt % was prepared, supplied to the first passage 11 by using the pump P1, and pushed into hexane flowing through the second passage 12 via the holes of the intermediate plate 3, so as to prepare a monodisperse W/O emulsion. FIG. 5 is a photomicrograph showing an enlarged view of this W/O emulsion.

This emulsion and a chitosan solution of 0.5 wt % (solvent: acetic acid) were put into a single vessel in a state of keeping the phase separation, and hexane was added to the emulsion.

By adding hexane, the emulsion was demulsified due to a decrease in the concentration of the surface-active agent. The carboxymethyl cellulose and the chitosan solution were brought into contact with respect to each other immediately, and polyelectrolyte complex gel was formed around the carboxymethyl cellulose droplets, so as to manufacture microcapsules of chitosan and carboxymethyl cellulose.

As mentioned above, by using the narrow holes (microchannels) formed in the plate (division wall), a monodisperse emulsion having a particle diameter of about 50 μm could be prepared. The capsules made from the emulsion were also monodisperse, that is, the diameter of the capsules had substantially the same particle diameter.

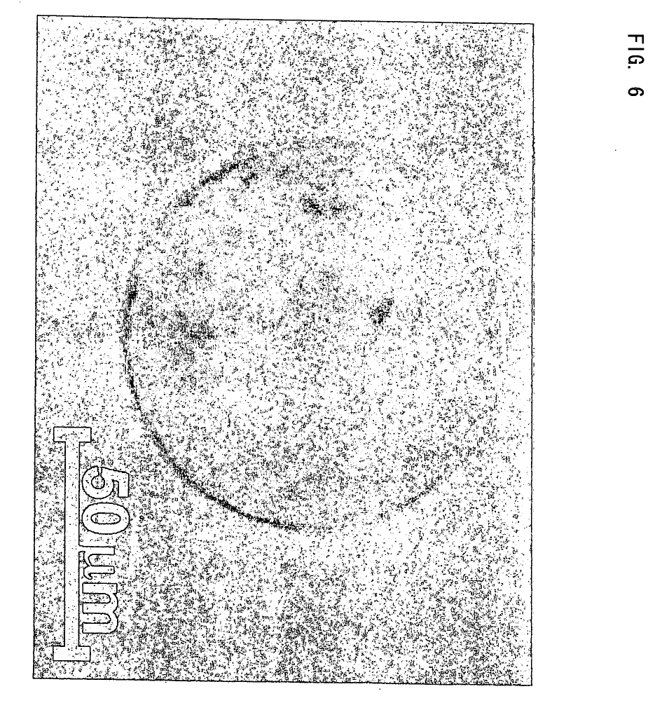

The preparation of the manufactured microcapsules was observed by a microscope, and the state where the surface film of the capsule was comprised of countless gel fibers was observed as shown in FIG. 6.

### EXAMPLE 2

An alginic acid (manufactured by KIMICA Corporation) was used as a raw material of the capsule. Soybean oil was used for an oil phase. An aqueous solution including a 0.1 M calcium chloride solution was used for a reaction solution.

An aqueous solution of an alginic acid of 1.5% (disperse phase) was supplied to the first passage 11, and soybean oil (continuous phase) in which no surface-active agent was added was supplied to the second passage 12. The aqueous solution of an alginic acid was pushed into the soybean oil via the holes (microchannels), so as to prepare an emulsion.

This emulsion was brought into contact with an aqueous solution of calcium chloride (polyvalent ion). As a result of this, capsules of calcium alginate were obtained.

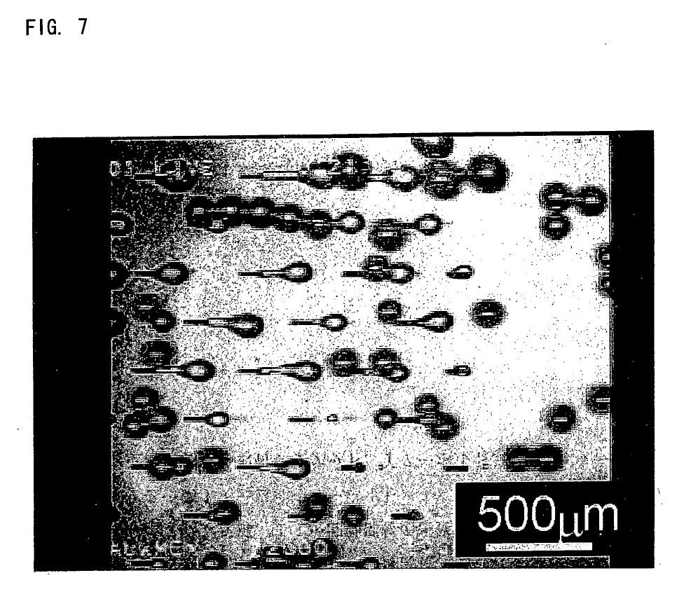

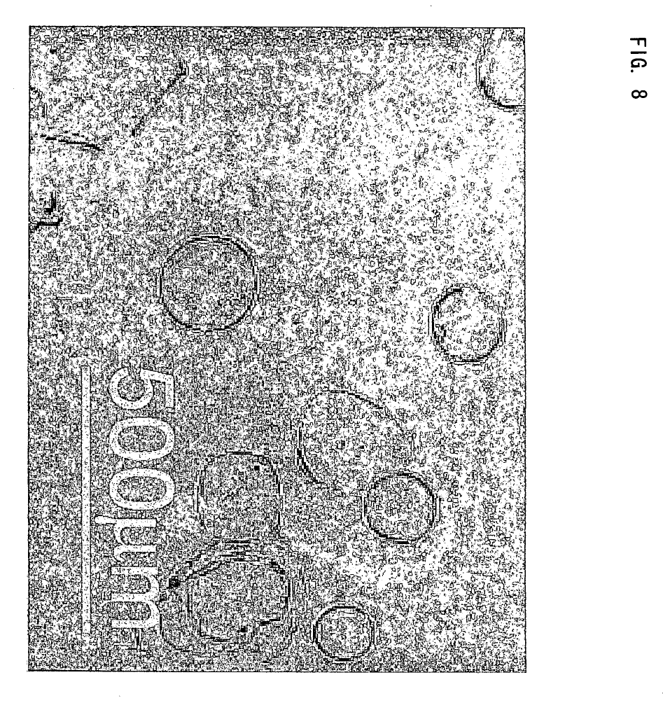

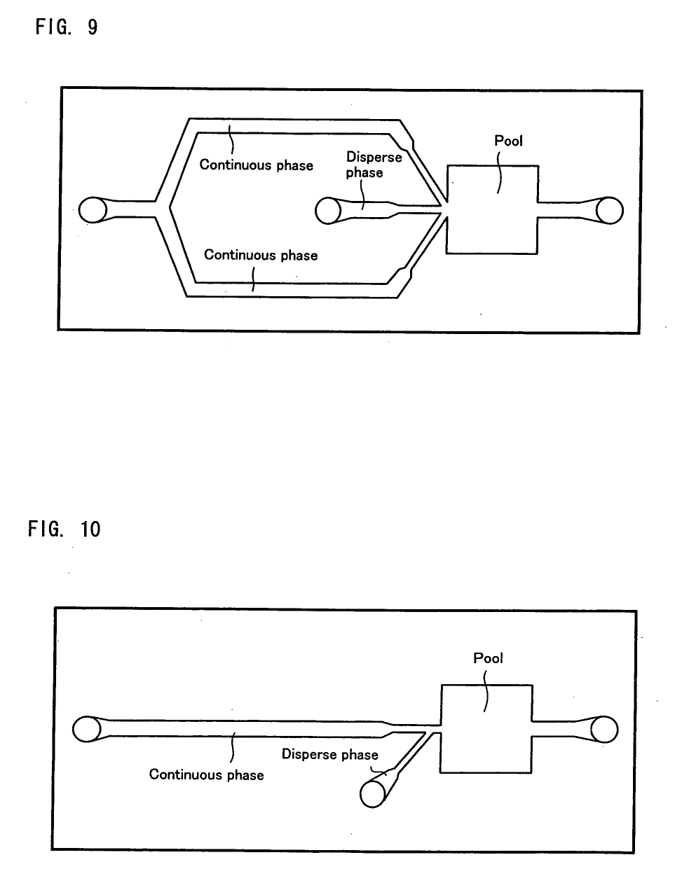

According to Example 2, as shown in FIG. 7, the obtained emulsion was homogenous, and the particle diameter of the disperse phase (droplet) was about 80 μm. This emulsion was contacted with the aqueous solution of calcium chloride, and thereby capsules having a particle diameter of around 100 μm were obtained.

In the apparatus used in the above-mentioned examples, after the emulsion was prepared, the disperse phase of the emulsion and a polyelectrolyte solution having a reverse electric charge or a polyvalent ion solution were contacted with respect to each other within another vessel so as to manufacture microcapsules. However, it is also possible to manufacture microcapsules in a single apparatus.

For example, a division wall may be provided in a substantially central area of the first passage 11 to divide the first passage into left and right sections. In this case, a disperse phase is supplied to the left section of the first passage by the pump P1 in the same manner as usual, and a polyelectrolyte solution having a reverse electric charge or a polyvalent ion solution is supplied to the right section of the first passage by another pump. With this, an emulsion is manufactured in an area on the upstream side of the second passage 12 where the disperse phase is supplied via the holes of the plate 3, and microcapsules are manufactured in an area on the downstream side (the right side of the drawing) where a polyelectrolyte solution having a reverse electric charge or a polyvalent ion solution is supplied via the holes of the plate 3.

In the above-mentioned method in which the disperse phase is introduced into the continuous phase via the narrow holes penetrating the thickness direction of the plate 3, the particle diameter of the disperse phase particles (microcapsules) in the emulsion depends on the diameter of the holes, and it is difficult to adjust the particle diameter.

In order to overcome this problem, there is another way to manufacture an emulsion which does not use narrow holes. Specifically, by allowing a continuous phase to flow through a microchannel, and a disperse phase to flow through another microchannel, both of which join with each other, the continuous phase and the disperse phase are allowed to join in a state of a laminar flow, and thereafter the flow rate of the continuous phase and the disperse phase are reduced in a dramatic way, so that the disperse phase particles can appear in the continuous phase. In this case, the disperse phase is taken into the continuous phase per one particle by a shearing stress, and the particle diameter can be controlled by adjusting the flow rate of the continuous phase and the disperse phase.

The microchannels are formed on a glass base or a silicon base. As a means for allowing the continuous phase and the disperse phase to join, the passages of the continuous phase may be arranged to join with the passage of the disperse phase from the both sides at an angle of 30-80°. Also, as a means for reducing the flow rate in a dramatic way, a pool having a large volume of capacity may be provided.

As mentioned above, according to the present invention, it is possible to stably produce a large quantity of capsules having a double structure in which a polyelectrolyte solution is encapsulated within a gel layer formed by a reaction between this polyelectrolyte solution and another polyelectrolyte solution in a state where the particle diameter is kept uniform.

Consequently, it is possible to obtain an effective capsule in a medical field such as for encapsulating a cell as well as in a food or cosmetic field.

### INDUSTRIAL APPLICABILITY

The present invention can effectively be used in a DDS (drug delivery system), a medical treatment for a human body, a food industry, or cosmetic manufacturing.

1-8. (canceled)

9. A manufacturing method for microcapsules comprising the steps of:
preparing an emulsion which contains a polyelectrolyte solution as a disperse phase having a uniform diameter according to the method of claim 19;
demulsifying the emulsion; and
contacting the polyelectrolyte solution as a disperse phase with a polyelectrolyte solution having a reverse electric charge to the polyelectrolyte solution as a disperse phase or a polyvalent ion solution at the same time as the demulsifying step so as to form a gel layer made of a polyelectrolyte complex around fine particles of the polyelectrolyte solution as a disperse phase by a polyelectrolyte reaction.

10. The manufacturing method for microcapsules according to claim 9, wherein the microchannels are formed on a glass base or a silicon base.

11. The manufacturing method for microcapsules according to claim 9, wherein the flow rate is reduced in a dramatic way by flowing the joined continuous and disperse phases into a pool having a large volume of capacity.

12. A manufacturing method for microcapsules, which is performed in a single apparatus comprising a case, a first passage for a disperse phase, a second passage for a continuous phase, a plate positioned between the first passage and the second passage, penetrating holes formed in the plate, and a division wall provided in a substantially central area of the first passage to divide the first passage into first and second sections, comprising the steps of:
supplying a continuous phase to the second passage;
supplying a polyelectrolyte solution as a disperse phase to the first section of the first passage in a state of applying greater pressure to the polyelectrolyte solution than to the continuous phase so as to push the disperse phase into the continuous phase via the penetrating holes to prepare an emulsion;
supplying a polyelectrolyte solution having a reverse electric charge to that of the polyelectrolyte solution as a disperse phase or a polyvalent ion solution to the second section of the first passage in a state of applying greater pressure to the polyelectrolyte solution having a reverse electric charge or the polyvalent ion solution than to the continuous phase; and
contacting the polyelectrolyte solution as a disperse phase with the polyelectrolyte solution having a reverse electric charge or the polyvalent ion solution while the emulsion is demulsified so as to form a gel layer made of a polyelectrolyte complex around fine particles of the polyelectrolyte solution as a disperse phase by a polyelectrolyte reaction.

13. The manufacturing method for microcapsules according to claim 9, wherein the emulsion is demulsified by adding the same material as the continuous phase or a material which is soluble in the continuous phase to the emulsion so as to reduce the concentration of a surface-active agent in the emulsion.

14. The manufacturing method for microcapsules according to claim 9, wherein the emulsion does not contain a surface-active agent and the emulsion is demulsified by being contacted with the polyelectrolyte solution having a reverse electric charge or the polyvalent ion solution.

15. The manufacturing method for microcapsules according to claim 9, wherein the disperse phase is selected from a group consisting of an alginic acid, carboxymethyl cellulose, pectin, carrageenan, sulfate cellulose, and chondroitin sulfuric acid; the polyelectrolyte to be reacted with the disperse phase is selected from a group consisting of a polyamino acid, polymer containing a primary amine group, a secondary amine group, a tertiary amine group, or pyridinyl nitrogen, and aminated polysaccharide; and the polyvalent ion in the polyvalent ion solution is selected from a group consisting of Ca²⁺, Ba²⁺, Pb²⁺, Cu²⁺, Cd²⁺, Sr²⁺, Co²⁺, Ni²⁺ and Mn²⁺.

16. The manufacturing method for microcapsules according to claim 9, wherein a cell which generates a desired material is added to the polyelectrolyte solution as a disperse phase in advance of the emulsion preparation step.

17. The manufacturing method for microcapsules according to claim 9, wherein the diameter of the disperse phase is within the range of 50-300 μm.

18. A method for treating a human body, wherein the microcapsule manufactured by the method according to claim 9 is injected into parts of a human body by an injector, a catheter or an operation.

19. A method for preparing an emulsion comprising the steps of: allowing a continuous phase material to flow through a microchannel;
allowing a polyelectrolyte solution as a disperse phase to flow through another microchannel, the microchannels being joined with each other to allow the continuous phase and the disperse phase to join in a state of a laminar flow; and
thereafter reducing the flow rate of the continuous phase and the disperse phase in a dramatic way so as to prepare an emulsion which contains the polyelectrolyte solution as a disperse phase having a uniform diameter.

20. A manufacturing method for microcapsules comprising the steps of:
preparing an emulsion which contains a polyelectrolyte solution as a disperse phase having a uniform diameter and a continuous phase;
demulsifying the emulsion; and
contacting the polyelectrolyte solution as a disperse phase with a polyelectrolyte solution having a reverse electric charge to the polyelectrolyte solution as a disperse phase or a polyvalent ion solution at the same time as the demulsifying step so as to form a gel layer made of a polyelectrolyte complex around fine particles of the polyelectrolyte solution as a disperse phase by a polyelectrolyte reaction.

21. The manufacturing method for microcapsules according to claim 20, wherein the emulsion is prepared by separately feeding the disperse phase and the continuous phase with a plate having penetrating holes, and applying greater pressure to the disperse phase than to the continuous phase so as to push the disperse phase into the continuous phase as microspheres.

22. The manufacturing method for microcapsules according to claim 20, wherein the emulsion is demulsified by adding the same material as the continuous phase or a material which is soluble in the continuous phase to the emulsion so as to reduce the concentration of a surface-active agent in the emulsion.

23. The manufacturing method for microcapsules according to claim 20, wherein the emulsion does not contain a surface-active agent, and the emulsion is demulsified by being contacted with the polyelectrolyte solution having a reverse electric charge to the polyelectrolyte solution as a disperse phase or the polyvalent ion solution.

24. The manufacturing method for microcapsules according to claim 20, wherein the disperse phase is selected from a group consisting of an alginic acid, carboxymethyl cellulose, pectin, carrageenan, sulfate cellulose, and chondroitin sulfuric acid; the polyelectrolyte to be reacted with the disperse phase is selected from a group consisting of a polyamino acid, polymer containing a primary amine group, a secondary amine group, a tertiary amine group, or pyridinyl nitrogen, and aminated polysaccharide; and the polyvalent ion of the polyvalent ion solution is selected from a group consisting of Ca²⁺, Ba²⁺, Pb²⁺, Cu²⁺, Cd²⁺, Sr²⁺, Co²⁺, Ni²⁺ and Mn²⁺.

25. The manufacturing method for microcapsules according to claim 20, wherein a cell which generates a desired material is added to the polyelectrolyte solution as a disperse phase in advance of said emulsion preparation step.

26. The manufacturing method for microcapsules according to claim 20, wherein the diameter of the disperse phase is within the range of 50-300 μm.

27. A method for treating a human body, wherein the microcapsule manufactured by the method according to claim 20 is injected into parts of a human body by an injector, a catheter or an operation.
